(19) United States
(12) Patent Application Publication
(10) Pub. No.: US 2012/0017918 A1
(43) Pub. Date: Jan. 26, 2012

(54) Oral Fixation Device and Fixing Method Using the Same

(75) Inventors: Chen-Ning Huang, Dayuan Township (TW); Chen-Liang Lin, Chiayi City (TW); Yunn-Jy Chen, Taipei City (TW)

(73) Assignees: NATIONAL TAIWAN UNIVERSITY HOSPITAL, Taipei (TW); INDUSTRIAL TECHNOLOGY RESEARCH INSTITUTE, Hsinchu (TW)

(21) Appl. No.: 12/977,293

(22) Filed: Dec. 23, 2010

(30) Foreign Application Priority Data
Jul. 21, 2010 (TW) 99124051

(51) Int. Cl. A61F 5/56; A61C 7/36

(52) U.S. Cl. 128/848; 433/19

(57) ABSTRACT

An oral fixation device and an oral fixing method using the same are provided. The oral fixation device includes a U-shaped brace and an arch board. The arch board is connected to an inner wall of the U-shaped brace, wherein the central part of the arch board is thicker than the connecting part, and the maximum thickness of the arch board is larger than 2 mm.

This application claims the benefit of Taiwan application Serial No. 99124051, filed Jul. 21, 2010, the subject matter of which is incorporated herein by reference.

### BACKGROUND

1. Technical Field

The disclosure relates in general to a fixing device and a fixing method using the same, and more particularly to an oral fixation device and an oral fixing method using the same.

2. Description of the Related Art

Nowadays, people need the oral fixation device for various reasons. However, the oral fixation device currently available can fix the teeth only but not the tongue at the same time.

Therefore, how to modify the current oral fixation device as a device which can fix the teeth, the tongue and other oral organs has become an imminent task for the industries.

### SUMMARY

The disclosure is directed to an oral fixation device and an oral fixing method using the same. The arch board and the U-shaped brace are combined, so that the user can fix the teeth as well as the tongue with the oral fixation device.

According to a first aspect of the present disclosure, an oral fixation device is provided. The oral fixation device includes a U-shaped brace and an arch board. The arch board is connected to an inner wall of the U-shaped brace, wherein the central part of the arch board is thicker than the connecting part, and the maximum thickness of the arch board is larger than 2 mm.

According to a second aspect of the present disclosure, an oral fixation device is provided. The oral fixation device includes a U-shaped brace and an arch board. The U-shaped brace is used for mounting an upper tooth and a lower tooth. The arch board is connected to an inner wall of the U-shaped brace and interposed between a tongue and a maxillary, wherein the maximum thickness of the arch board is substantially equal to the distance between the tongue and the maxillary.

According to a third aspect of the present disclosure, an oral fixing method is provided. The oral fixing method includes the following steps. An oral fixation device is provided, wherein the oral fixation device includes a U-shaped brace and an arch board connected to an inner wall of the U-shaped brace. The oral fixation device is interposed between a tongue and a maxillary, so that an upper surface of the arch board substantially contacts the maxillary, and a lower surface of the arch board substantially contacts the tongue. An upper tooth and a lower tooth are disposed into the U-shaped brace.

The above and other aspects of the disclosure will become better understood with regard to the following detailed description of the non-limiting embodiment(s). The following description is made with reference to the accompanying drawings.

### BRIEF DESCRIPTION OF THE DRAWINGS

FIG. 1 is a 3-D diagram of an oral fixation device according to an embodiment of the disclosure;

FIG. 2 is a back view of an oral fixation device;

FIG. 3 is an upper view of an oral fixation device;

FIG. 4 is a cross-sectional view of the oral fixation device of FIG. 3 viewed along a cross-sectional line 4-4′;

FIG. 5 is a flowchart of using an oral fixation device; and

FIG. 6 is a user interposing the oral fixation device between the tongue and the maxillary.

### DETAILED DESCRIPTION

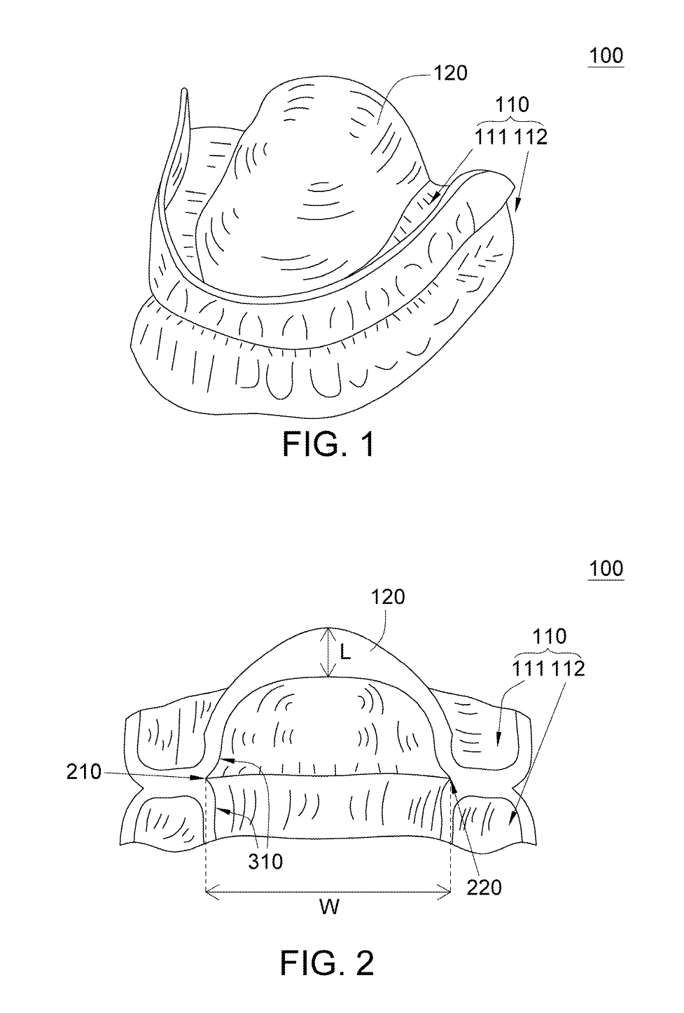

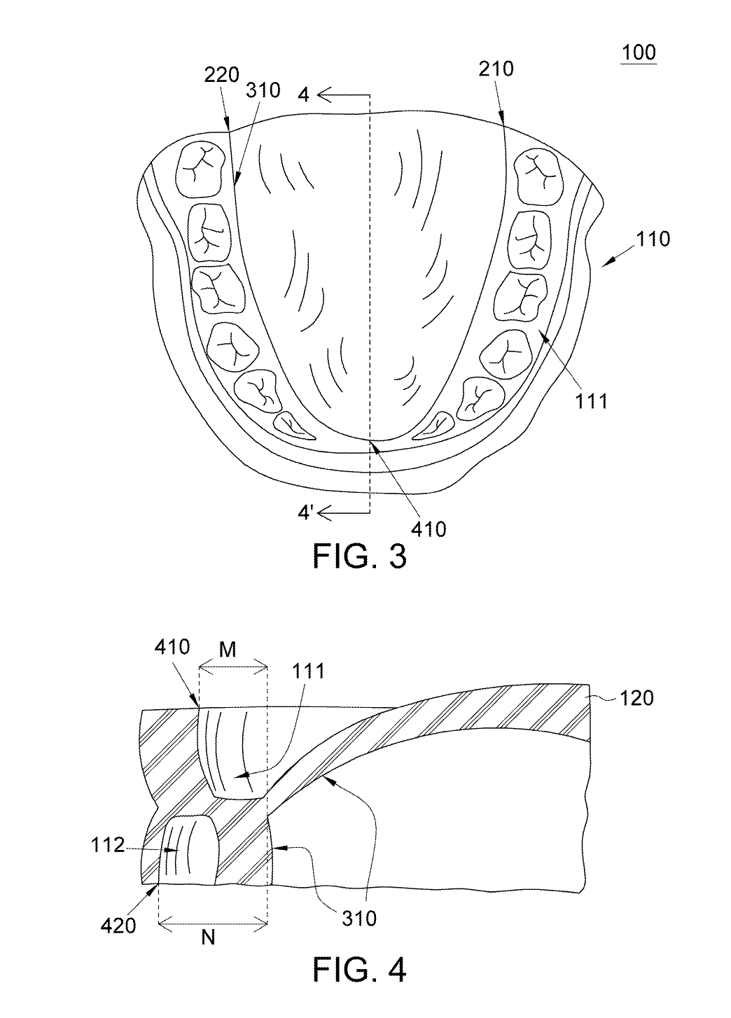

Referring to FIGS. 1˜4. FIG. 1 is a 3-D diagram of an oral fixation device 100 according to an embodiment of the disclosure. FIG. 2 is a back view of an oral fixation device 100. FIG. 3 is an upper view of an oral fixation device 100. FIG. 4 is a cross-sectional view of the oral fixation device of FIG. 3 viewed along a cross-sectional line 4-4′.

The oral fixation device 100 of the present embodiment of the disclosure includes a U-shaped brace 110 and an arch board 120. The U-shaped brace 110 is used for mounting an upper tooth 630 (illustrated in FIG. 6) and a lower tooth 640 (illustrated in FIG. 6) of an oral cavity. The arch board 120 with non-uniform thickness is connected to an inner wall 310 of the U-shaped brace 110 and interposed between a tongue 610 (illustrated in FIG. 6) and a maxillary 620 (illustrated in FIG. 6). The central part of the arch board 120 is thicker than the connecting part. For example, the thickness of the arch board 120 progressively increases from the connecting part towards the central part. In an embodiment of the disclosure, the U-shaped brace can be a bite block.

As indicated in FIG. 2, a maximum thickness L of the arch board 120 being substantially equal to the distance between a tongue 610 and a maxillary 620 is larger than 2 mm but smaller than 15 mm or is tailor made to fit the user's needs. For example, if the user is a child, the maximum thickness L of the arch board 120 can range between 2˜8 mm, so that the arch board 120 can hold the tongue 610 and the cause of respiratory tract obstruction can be removed.

As indicated in the oral device 100 of FIG. 4, the U-shaped brace 110 further has a U-shaped upper bite channel 111 and a U-shaped lower bite channel 112. The front end 410 of the U-shaped upper bite channel 111 is separated from the inner wall 310 by a first distance M. The front end 420 of the U-shaped lower bite channel 112 is separated from the inner wall 310 by a second distance N. The second distance N is larger than the first distance M. In other words, the U-shaped lower bite channel 112 is substantially located in front of the U-shaped upper bite channel 111, and when the user wears the oral fixation device 100, the mandible 650 (illustrated in FIG. 6) will be slightly advanced forwards, so that the cross area of the respiratory tract can be enlarge.

In addition, the arch board 120 is interposed between a tongue 610 and a maxillary 620. To stabilize the position of the user's tongue, the arch board 120 fills the space between the tongue 610 and the maxillary 620 of the user's oral cavity and characterizes a non-uniform distribution of thickness. Again, referring to FIG. 2, the maximum thickness L of the arch board 120 is located at the central part of the arch board 120. That is, the arch board 120 is thicker at the central part but thinner at the two sides. In the oral cavity of a human, the clearance between the tongue 610 and the maxillary 620 is also wider at the central part and narrower at the two sides. When the user wears the oral fixation device 100, the above design enables the user's tongue to be firmly fixed under the arch board 120 of the oral fixation device 100.

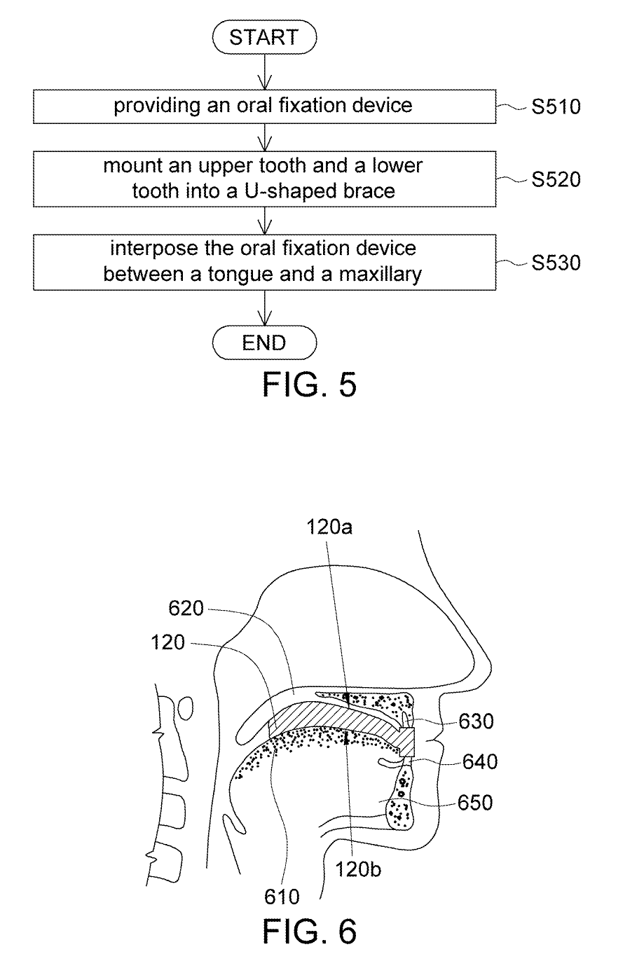

Referring to FIG. 5 and FIG. 6. FIG. 5 is a flowchart of using an oral fixation device 100. FIG. 6 is the user interposing an oral fixation device 100 between a tongue 610 and a maxillary 620. Firstly, the method begins at step S510, an oral fixation device 100 is provided.

Next, the method proceeds to step S520, an upper tooth 630 and a lower tooth 640 are mounted into a U-shaped brace 110. As indicated in FIG. 6, the upper tooth 630 and the lower tooth 640 are firmly fixed in the U-shaped brace 110, and the biting position of the upper tooth and the lower tooth is adjusted, so that the front end 410 of the U-shaped upper bite channel 111 is separated from the inner wall 310 by a first distance M, and the front end 420 of the U-shaped lower bite channel 112 is separated from the inner wall 310 by a second distance N which is larger than the first distance M. In other words, the U-shaped lower bite channel 112 is substantially located in front of the U-shaped upper bite channel 111 as indicated in FIG. 4.

Then, the method proceeds to step S530, the oral fixation device 100 is interposed between the tongue 610 and the maxillary 620, and the thickness of the arch board 120 is adjusted according to the clearance between the tongue 610 and the maxillary 620, so that an upper surface 120a of the arch board 120 substantially contacts the maxillary 620, and a lower surface 120b of the arch board 120 substantially contacts the tongue 610. It is shown in FIG. 6 that when the upper surface 120a and the lower surface 120b of the arch board 120 substantially contact the maxillary 620 and the tongue 610 respectively, the tongue 610, being squeezed by the arch board 120 which is fixed under the maxillary 620, is fixed under the arch board 120. Thus, when the user's tooth bites the U-shaped brace 110, the arch board 120 is concurrently squeezed by both the U-shaped brace 110 and the maxillary 620, the arch board 120 fixes the tongue 610 from above, front, the left and the right to assure that the tongue 610 is firmly fixed.

According to the design of the oral fixation device 100 of the present embodiment of the disclosure, the front end 410 of the U-shaped upper bite channel 111 is separated from the inner wall 310 by a first distance M, and the front end 420 of the U-shaped lower bite channel 112 is separated from the inner wall 310 by a second distance N which is larger than the first distance M, so the oral fixation device 100 can maintain the open of the respiratory tract as the mandible 650 (illustrated in FIG. 6) is advanced forward. Moreover, since the arch board 120 can hold the tongue 610, the cause of respiratory tract obstruction can be removed. In addition, the arch board 120 fills the space surrounding the tongue 610, supports the tongue 610 and prevents it from falling due to gravity when the user lies down. Thus, the user's respiratory tract will not be obstructed. Therefore, the oral fixation device 100 of the present embodiment of the disclosure at least has the features of “maintaining the open of the respiratory tract”, “removing the cause of respiratory tract obstruction” and “keeping the tongue at a natural position lest the tongue might fall due to gravity”.

The tongue 610 is kept at a natural position and can be easily stabilized by the oral fixation device 100 with only a small amount of effort, so the user will not feel uncomfortable even after wearing the oral fixation device 100 for a long duration. Since the oral fixation device 100 can fix the upper tooth 630 and the lower tooth 640, the user's mouth will not be opened to breathe when he is asleep with the oral fixation device 100 in his oral cavity. The oral fixation device 100 of the present embodiment of the disclosure further has the feature of “causing no discomfort even after a long duration of use”.

Experiments were undertaken with five testees from July 2009 to December 2009 to assess the effect of oral fixation device of present disclosure. At first, these five testees were checked that they are not the following patients. (1) Non-obstructive sleep apnea patients. (2) Patients having other disease about sleeping and breathing except obstructive sleep apnea syndrome (OSAS). (3) Patients having systemic disease, such as rheumatoid arthritis. (4) Patients having Temporo-Mandibular disorder. (5) Patients having periodontal disease or toothache, or people who can't use braces.

The experiment was started at the step of making testees to undergo polysomnography (PSG) examination, recording the age, body mass index (BMI), and neck circumference data of the testees, and taking a lateral cephalometric photography for testees. Then dentist produced a general oral appliance and an oral fixation device of the present disclosure separately for the testees after modeling the oral cavity. The testees wore the general oral appliances three months and underwent PSG examination to assess the effect of wearing the general oral appliances. And then the testees wore the oral fixation device of the present disclosure three months and underwent PSG examination to assess the effect of wearing the oral fixation device. Table 1 shows the result of the experiment. Wherein the “AHI” means the apnea hypopnea index (the severity of OSAS)

TABLE 1
The result of the experiment

| | | AHI of 2^{nd} PSG | AHI of 3^{rd} PSG | Improve- |
|---|---|---|---|---|
| Test- | AHI of 1^{st} PSG | Wear General | Wear the Oral | ment AHI |
| ee | Before Test | Oral Appliance | Fixation Device | ΔAHI |
| A | 14.8 | 16.5 | 1.5 | 89.56% |
| B | 116.3 | 82.2 | 67.7 | 41.79% |
| C | 57.6 | 58.0 | 45.6 | 20.83% |
| D | 9.2 | 18.9 | 1.3 | 85.87% |
| E | 25.1 | 51.8 | 10.4 | 58.57% |

It is clear that testee wearing the oral fixation device of present disclosure would reduce the severity of OSAS. Thus, in contrast to the general oral appliances, the oral fixation device of the disclosure is more suitable for being used as an oral appliance for preventing respiratory tract obstruction, which may cause sleep apnea.

While the disclosure has been described by way of example and in terms of the exemplary embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. An oral fixation device, comprising:
a U-shaped brace; and
an arch board connected to an inner wall of the U-shaped brace, wherein the central part of the arch board is thicker than the connecting part, and the maximum thickness of the arch board is larger than 2 mm.

2. The oral fixation device according to claim 1, wherein the maximum thickness of the arch board is smaller than 15 mm.

3. The oral fixation device according to claim 1, wherein the U-shaped brace has a U-shaped upper bite channel and a U-shaped lower bite channel, the front end of the U-shaped upper bite channel is separated from the inner wall by a first distance, the front end of the U-shaped lower bite channel is separated from the inner wall by a second distance, and the second distance is larger than the first distance.

4. The oral fixation device according to claim 1, wherein the maximum thickness is located at the central part of the arch board.

5. The oral fixation device according to claim 1, wherein the maximum thickness is larger than 2 mm but smaller than 8 mm.

6. The oral fixation device according to claim 1, wherein the thickness of the arch board increases progressively from the connecting part towards the central part.

7. An oral fixation device, comprising:
a U-shaped brace used for mounting an upper tooth and a lower tooth; and
an arch board connected to an inner wall of the U-shaped brace and interposed between a tongue and a maxillary, wherein a thickness of the arch board is substantially equal to the distance between the tongue and the maxillary.

8. The oral fixation device according to claim 7, wherein the U-shaped brace further comprises a U-shaped upper bite channel and a U-shaped lower bite channel, the front end of the U-shaped upper bite channel is separated from the inner wall by a first distance, the U-shaped front end of the U-shaped lower bite channel is separated from the inner wall by a second distance, and the second distance is larger than the first distance.

9. An oral fixing method, comprising:
providing an oral fixation device, which comprises a U-shaped brace and an arch board connected to an inner wall of the U-shaped brace;
disposing an upper tooth and a lower tooth into the U-shaped brace; and
interposing the oral fixation device between a tongue and a maxillary, so that an upper surface of the arch board substantially contacts the maxillary, and a lower surface of the arch board substantially contacts the tongue.

10. The method according to claim 9, wherein in the step of providing the oral fixation device, the U-shaped brace further comprises a U-shaped upper bite channel and a U-shaped lower bite channel, the front end of the U-shaped upper bite channel is separated from the inner wall by a first distance, the U-shaped front end of the U-shaped lower bite channel is separated from the inner wall by a second distance, and the second distance is larger than the first distance.
